(19) United States
(12) Patent Application Publication
(10) Pub. No.: US 2021/0221758 A1
(43) Pub. Date: Jul. 22, 2021

(54) A PROCESS FOR METHANOL PRODUCTION USING A LOW-IRON CATALYST

(71) Applicant: HALDOR TOPSØE A/S, Kgs. Lyngby (DK)

(72) Inventors: Søren Grønborg ESKESEN, Espergærde (DK); Per Juul DAHL, Vedbæk (DK); Emil Andreas TJÄRNEHOV, Limhamn (SE); Max THORHAUGE, Herlev (DK)

(73) Assignee: HALDOR TOPSØE A/S, Kgs. Lyngby (DK)

(21) Appl. No.: 17/055,399

(22) PCT Filed: Jun. 11, 2019
(86) PCT No.: PCT/EP2019/065132

§ 371 Date: Nov. 13, 2020

(30) Foreign Application Priority Data
Jun. 12, 2018 (DK) PA 2018 00268

(51) Int. Cl. C07C 29/154; B01J 21/04; B01J 23/80; B01J 19/18

(52) U.S. Cl. CPC C07C 29/154; B01J 21/04; C07C 31/04; B01J 19/1837; B01J 23/80

(57) ABSTRACT

The deterioration of methanol synthesis catalysts that is caused by iron poisoning of the catalyst is counteracted by using a catalyst containing a maximum of 100 ppmw Fe in the synthesis process. The method is especially useful in a methanol synthesis plant comprising a make-up gas compressor and a synthesis reactor in a methanol loop with a once-through pre-converter installed between the make-up gas compressor and the methanol loop.

The present invention relates to means for counteracting the deterioration of methanol synthesis catalysts that is caused by iron poisoning of the catalyst. More specifically, the invention concerns optimal operating conditions for avoiding poisoning of methanol synthesis catalysts.

Methanol is synthesized from synthesis gas (syngas), which consists of H₂, CO and CO₂. The conversion from syngas is performed over a catalyst, which is most often a copper-zinc oxide-alumina (Cu/ZnO/Al₂O₃) catalyst. The methanol synthesis by conversion from syngas can be formulated as a hydrogenation of carbon dioxide, accompanied by the shift reaction, and it can be summarized by the following reaction sequence comprising the reactions (1)-(3) below:

CO+2H₂<->CH₃OH (1)

CO₂+3H₂<->CH₃OH+H₂O (2)

CO+H₂O<->CO₂+H₂ (3)

of which reaction (3) is the water-gas shift (WGS) reaction. The synthesis reaction occurring on the copper metal surface of the Cu/ZnO/Al₂O₃ catalyst is predominantly reaction (2), i.e. the formation of methanol from carbon dioxide. While such aspects of methanol synthesis catalysis as the kinetics and mechanism of reaction and the nature of catalytically active sites have been the subject of several investigations over the last decades, the literature on the deactivation of methanol synthesis catalysts is, in contrast, relatively sparse. An exception is a 1992 review of the methanol catalyst deactivation by H. H. Kung (Catalysis Today 92 (1992), 443), which focuses on the issue of sulfur poisoning, whereas deactivation by iron is only mentioned in the sense that deposition of iron on the catalyst surface may block the active sites and also provide undesired catalytic activities, such as forming hydrocarbons by the Fischer-Tropsch reaction, which then becomes a competing reaction.

The activity of the Cu/ZnO/Al₂O₃ methanol catalyst is directly related to the copper surface area of the material. Therefore, manufacture of the catalyst requires the preparation of phases that will give high and stable copper surface areas. During operation in real methanol plants, three main deactivation processes may take place on methanol synthesis catalysts: Thermal sintering, catalyst poisoning and reactant-induced deactivation. The thermal sintering is a temperature-induced loss of copper surface area with time, the catalyst poisoning is transport of catalyst poisons into the methanol converter with the process gas, and the reactant-induced deactivation is a deactivation caused by the composition of the reactant gases. These deactivation processes will all lead to a permanent loss of catalyst activity, and in the end, poisoning of the catalyst will lead to a permanent loss of catalyst selectivity.

This invention especially deals with methanol catalyst poisoning caused by iron, originating from the metal parts of the plant transported into the methanol converter with the process gas. The iron is transported into the converter as a volatile iron species Fe(CO)₅ (iron pentacarbonyl or just iron carbonyl), which is generated by low-temperature reaction of CO-rich gas with metal surfaces in other parts of the plant. However, at more elevated temperatures, such as those found in the synthesis converter, the iron carbonyl will readily decompose upon contact with the high surface area copper catalyst. Unlike poisoning with sulfur (for which the impact on the activity can be reduced in cases where the catalyst has been formulated in such a way that the zinc oxide component is allowed to act as an absorbent for the sulfur poison), there is no natural absorbent effect for iron within the Cu/ZnO/Al₂O₃ catalyst (Ind. Eng. Chem. Res. 32, 1993, pg. 1610-1621).

Regarding thermal sintering, temperature is the dominant factor in controlling the rate of sintering of metallic and oxidic species. Copper has a relatively low melting point (1083° C.) compared to other commonly used metallic catalysts such as iron (1535° C.) and nickel (1455° C.)

A large number of materials exist, which in principle could act as poisons on a Cu/ZnO/Al₂O₃ catalyst, but only a few of these are regularly discovered upon analysis of discharged catalyst samples. For example, silica (which would lower the synthesis activity and promote by-product formation) and chloride (which causes very high rates of copper crystallite sintering) are both poisons for copper catalysts, but they are rarely transported onto the synthesis catalyst in any significant quantities in well-operated methanol plants. However, besides nickel and sulfur, especially iron (having been brought into the converter as iron carbonyl as described above) is often found in significant quantities on discharged methanol synthesis catalysts. In addition to poisoning the catalyst, the presence of iron within the methanol plant has the effect that methane, paraffins and detrimental long-chained waxes are formed.

It has now been found by the Applicant that, in order to avoid deactivation of the Cu/ZnO/Al₂O₃ methanol catalyst, an optimal condition is to use a catalyst having a content of maximum 100 ppmw Fe. Using a catalyst containing more than 100 ppmw Fe will lead to a fast catalyst deactivation. This goes for the use of the catalyst in any plant design or any layout around the methanol reactor, such as the methanol loop with or without pre-converter and irrespective of whether the layout is a novel design or a revamp.

A typical methanol plant operated with a natural gas feed is divided into three main sections. In the first part of the plant, natural gas is converted into syngas. The syngas reacts to produce methanol in the second section, and then methanol is purified to the desired purity in the tail-end of the plant. In a standard synthesis loop, a methanol reactor, most often a boiling-water reactor (BWR), is used to convert a mixture of synthesis gas from a reformer/gasifier unit and recycle gas, i.e. unconverted synthesis gas, into methanol.

So the present invention concerns a process for the production of methanol from synthesis gas via an equilibrium reaction proceeding at elevated temperatures under elevated pressure according to the above synthesis reactions (1) to (3), said process being conducted by using a catalyst containing a maximum of 100 ppmw Fe.

In the prior art, iron contaminants in a hydrocarbon feedstock have been shown to poison the catalyst and reduce its activity. Thus, EP 3 052 232 B1 relates to a process for reactivating an iron-contaminated FCC (fluid catalytic cracking) catalyst. The poisoning occurs when iron clogs the surface of the catalyst, which (besides the poisoning) results in a significant decrease in apparent bulk density of the catalyst. According to the EP document, an iron transfer agent that comprises a magnesia-alumina hydrotalcite material is used for reactivating the FCC catalyst.

In U.S. Pat. No. 9,314,774 B1, an attempt is made to postpone the deactivation of the Cu/ZnO/Al₂O₃ catalyst by using a catalyst with a very specific composition, i.e. a Zn/Cu molar ratio of 0.5 to 0.7, a Si/Cu molar ratio of 0.015 to 0.05, a maximum intensity ratio of a peak derived from zinc to a peak derived from copper of not more than 0.25 and a half-value width (2θ) of the peak derived from copper of 0.75 to 2.5. Further, said catalyst may have a zirconium content of up to 0.01 mol %.

US 2012/0322651 A1 describes a multistage process for preparing methanol, comprising a plurality of serial synthesis stages, in which the severity of the reaction conditions, based on the reaction temperature and/or the concentration of carbon monoxide in the synthesis gas, decreases from the first to the last reaction stage in the flow direction. The first reaction stage has a first catalyst of low activity, but high long-term stability, while the last reaction stage has a second catalyst of high activity, but low long-term stability. Only a partial conversion of synthesis gas to methanol is achieved per passage through each reaction stage, and therefore recirculation of non-converted synthesis gas to the reaction stages is necessary.

A method for producing methanol from inert-rich syngas is disclosed in US 2014/0031438 A1. A catalytic pre-reactor is installed upstream of the synthesis loop, a first part of the syngas being converted to methanol in the catalytic pre-reactor. Furthermore, an inert gas separation stage, e.g. a PSA system or a membrane system, is connected downstream of the synthesis loop, whereby a hydrogen-enriched syngas stream can be returned to the synthesis loop. In the processing of methane-rich syngas, the inert gas separation stage may also comprise an autothermal reformer in which methane is converted to carbon oxides and hydrogen, which are also returned into the synthesis loop.

In Applicant's WO 2017/025272 A1, a process for methanol production from low quality synthesis gas is described, in which relatively smaller adiabatic reactors can be operated more efficiently, whereby some of the disadvantages of adiabatic reactors for methanol production are avoided. This is done by controlling the outlet temperature in the pre-converter by rapid adjustment of the recycle gas, i.e. by manipulating the gas hourly space velocity in the pre-converter.

A combined anaerobic digester and gas-to-liquid system is disclosed in WO 2016/179476 A1. The anaerobic digester requires heat and produces methane, and the gas-to-liquid system converts methane to higher value products, including methanol and formaldehyde.

It is well known in the art that a synthesis gas derived from natural gas or heavier hydrocarbons and coal is highly reactive for direct methanol synthesis and harmful for the catalyst. Moreover, use of such highly reactive synthesis gas results in formation of large amounts of by-products.

The reaction of carbon oxides and hydrogen to methanol is equilibrium-limited, and the conversion of the synthesis gas to methanol per pass through the methanol catalyst is relatively low, even when using a highly reactive synthesis gas.

Because of the low methanol production yield in a once-through conversion process, the general practice in the art is to recycle unconverted synthesis gas separated from the reaction effluent and dilute the fresh synthesis gas with the recycle gas.

This typically results in the so-called methanol synthesis loop with one or more reactors connected in series being operated on fresh synthesis gas diluted with recycled unconverted gas separated from the reactor effluents or on the reactor effluent containing methanol and unconverted synthesis gas. The recycle ratio (recycle gas to fresh synthesis feed gas) is from 2:1 up to 7:1 in normal practice. If a pre-converter is installed between the make-up gas compressor and the methanol loop, then the pre-converter will catch the iron originating from the front-end. Even though the presence of iron as well as the partial pressure of CO and the temperature are known to have an impact of formation of long-chained wax, the mechanisms and limits are not entirely understood.

As for the catalyst itself, it has been calculated that a Cu/ZnO/Al₂O₃ catalyst with a content of 100 ppmw Fe will have an expected life time of 4 years. The actual life time has turned out to be 4 years also.

For a Cu/ZnO/Al₂O₃ catalyst with a larger content of Fe, more specifically 1500 ppmw Fe, it has been calculated that the expected life time was 3 years. In this case, however, the actual life time turned out to be only 1.5 years, which is proof that a high iron content decreases the life time of the catalyst more than expected.

1. A process for the production of methanol from synthesis gas via an equilibrium reaction proceeding at elevated temperatures under elevated pressure according to the reactions
CO+2H₂<->CH₃OH (1)
CO₂+3H₂<->CH₃OH+H₂O (2)
CO+H₂O<->CO₂+H₂ (3)
said process being conducted by using a catalyst containing a maximum of 100 ppmw Fe.

2. Process according to claim 1, wherein the catalyst is a Cu/ZnO/Al₂O₃ methanol catalyst.

3. A plant for the production of methanol by the process according to claim 1, said plant comprising a make-up gas compressor and a synthesis reactor in a methanol loop
with a once-through pre-converter installed between the make-up gas compressor and the methanol loop, wherein a catalyst containing a maximum of 100 ppmw Fe is used.

4. A plant for the production of methanol by the process according to claim 2, said plant comprising a make-up gas compressor and a synthesis reactor in a methanol loop
with a once-through pre-converter installed between the make-up gas compressor and the methanol loop, wherein a catalyst containing a maximum of 100 ppmw Fe is used.
